(19) United States
(12) Patent Application Publication
(10) Pub. No.: US 2020/0261727 A1
(43) Pub. Date: Aug. 20, 2020

(54) MULTI-CHANNEL ELECTRIC STIMULATOR FOR NEURAL IMPLANT

(71) Applicant: TODOC CO., LTD., Seoul (KR)

(72) Inventors: Jeong Hoan PARK, Hanam-si (KR); Ho Seung LEE, Seoul (KR); Woo Jin AHN, Seoul (KR)

(21) Appl. No.: 16/279,316

(22) Filed: Feb. 19, 2019

(51) Int. Cl. A61N 1/36; A61N 1/20; A61N 1/02

(52) U.S. Cl. CPC A61N 1/36125; A61N 1/025; A61N 1/20

(57) ABSTRACT

A multi-channel electric stimulator for a neural implant switches direct current according to nerve stimulation channels of different sequences of nerve stimulation. A direct current generator supplies direct current in response to nerve stimulation in the sequence of stimulation by controlling a nerve stimulation channel next in the sequence to stand by, with an electric current of the next nerve stimulation channel being modified into a direct current level, when nerve stimulation is performed using a nerve stimulation channel earlier in the sequence of stimulation. A biphasic current pulse generator generates a biphasic current pulse by switching the nerve stimulation channel-specific direct current supplied thereto. A controller controls a switching operation of the biphasic current pulse generator depending on a sequence of stimulation set according to the nerve stimulation channels. DAC switching noise induced by the occurrence of a biphasic current pulse is not transferred to the circuit.

### BACKGROUND OF THE INVENTION

### Field of the Invention

The present disclosure relates generally to a multi-channel electric stimulator used in biomedical engineering, in particular, in neural prostheses, such as a deep brain stimulator, a cochlear prosthesis, and a retinal prosthesis, to stimulate a nerve using electric current.

### Description of the Related Art

The information disclosed in the Background of the Invention section is only for the enhancement of understanding of the background of the invention, and should not be taken as an acknowledgment or as any form of suggestion that this information forms a prior art that would already be known to those skilled in the art.

In general, a neural implant refers to an electronic device replacing the function of a damaged nerve or restricting undesirable neural activity by electrically stimulating the nerve. Such neural prostheses may include a cochlear prosthesis, a retinal prosthesis, a deep brain stimulator, and the like. A related-art is disclosed in Korean Patent Application No. 10-2007-0051150 (titled “Electric Stimulator”).

In such a neural implant, FIG. 1 is a circuit diagram illustrating a structure of a multi-channel electric stimulator of the related art, generally used for continuous interleaved sampling (CIS) stimulation. In such a multi-channel electric stimulator of the related art, n-type metal-oxide-semiconductor field-effect transistor (nMOSFET) switches S [5:0] act as a biphasic current pulse generator (BCG), and a single digital-to-analog converter (DAC) is shared. CIS stimulation means a stimulation strategy designed to minimize interference between channels, in which the multi-channel electric stimulator performs stimulation sequentially using channels such that nerve stimulation waveforms overlap in no sections, instead of performing stimulation simultaneously using all channels. The BCG is a circuit that converts direct current (DC), generated by the DAC, into a biphasic current pulse. The DAC is a circuit that determines the magnitude of current using a digital signal when generating a biphasic current pulse from a nerve stimulation waveform.

FIG. 2 illustrates a signal diagram of the multi-channel electric stimulator of the related art. The operation of the multi-channel electric stimulator of the related art will be described with reference to FIG. 2. First, in a case in which a biphasic current pulse is generated between a first channel CH1 and a nerve stimulation reference REF and then a biphasic current pulse is generated between a second channel CH2 and the nerve stimulation reference REF, when a current level is changed, noise in the form of a glitch occurs in voltages on both ends of the DAC during DAC switching. Such switch noise causes noise in the biphasic current pulses between the channels CH1 and CH2. In the signal diagram illustrated in FIG. 2, portions indicated by dotted lines mean cases without noise, while portions indicated by thick solid lines mean current noise caused by the DAC.

This phenomenon in the nerve stimulation is undesirable, since an accurate level of stimulation is important. Thus, to prevent this phenomenon in the nerve stimulation, a length of time, during which voltages on both ends of the DAC are stabilized, is provided by adding a DAC switching state. However, this solution may not be effective when applied to the multi-channel electric stimulator having a large number of channels, and a new multi-channel electric stimulator for a neural implant is demanded.

### RELATED ART DOCUMENT

Patent Document 1: Korean Patent Application No. 10-2007-0051150 (filed on May 26, 2007; titled “Electric Stimulator”)

### SUMMARY OF THE INVENTION

Accordingly, the present invention has been made keeping in mind the above problems occurring in the related art, and the present invention proposes a multi-channel electric stimulator for a neural implant. The multi-channel electric stimulator for a neural implant has a simple and effective structure, by which a biphasic current pulse can be prevented from having current noise induced digital-to-analog converter (DAC) switching noise when the multi-channel electric stimulator stimulates the nerve.

In order to achieve the above object, according to one aspect of the present invention, there is provided a multi-channel electric stimulator for a neural implant. The multi-channel electric stimulator supplies direct current to stimulate the nerve in the sequence of stimulation. When the nerve is stimulated using a nerve stimulation channel earlier in the sequence of stimulation among adjacent nerve stimulation channels, a nerve stimulation channel next in the sequence of stimulation among the adjacent nerve stimulation channels stands by, with an electric current thereof being modified into a direct current (DC) level.

More specifically, when none of two DACs connected to a multiplexer connected to a biphasic current pulse generator (BCG) are selected by the multiplexer and another pulse waveform is generated, electric current is modified to a current level to flow at a selected point in time. Accordingly, the magnitude of the electric current is modified before the standby operation, and the electric current is allowed to flow, with the magnitude thereof being determined, when a next stimulation pulse is generated.

According to embodiments, DAC switching noise induced by the occurrence of a biphasic current pulse is not transferred to the circuit, and thus no current noise induced by DAC switching noise is created in a biphasic current pulse.

In addition, it is possible to reduce stimulation pulse intervals as small as possible in the case of continuous interleaved sampling (CIS) stimulation, thereby providing an electric stimulation circuit having a higher pulse rate. Thus, it is possible to reduce inter-channel intervals, thereby performing CIS stimulation using more channels in a predetermined time.

Furthermore, the multi-channel electric stimulator can be effectively used in a cochlear prosthesis provided with a large number of channels.

### BRIEF DESCRIPTION OF THE DRAWINGS

The above and other objects, features and advantages of the present invention will be more clearly understood from the following detailed description when taken in conjunction with the accompanying drawings, in which:

FIG. 1 is a circuit diagram illustrating a structure of a multi-channel electric stimulator for a neural implant of the related art;

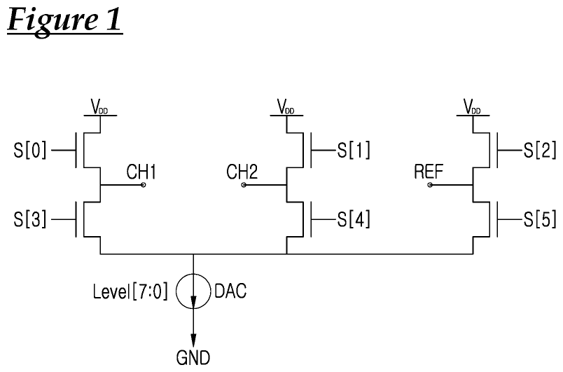

FIG. 2 illustrates a signal diagram of the multi-channel electric stimulator for a neural implant of the related art;

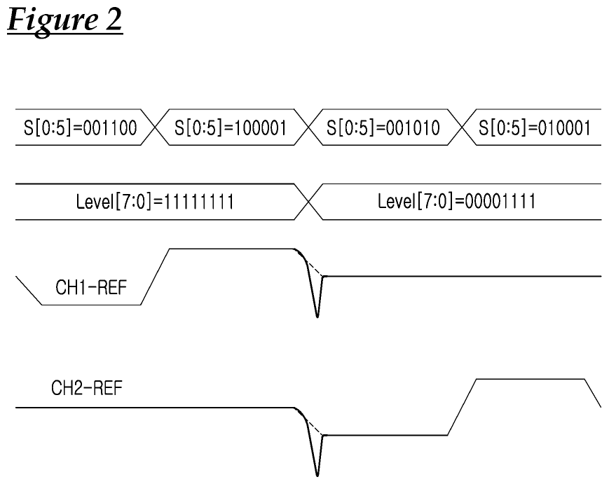

FIG. 3 is a circuit diagram illustrating a structure of a multi-channel electric stimulator for a neural implant according to an exemplary embodiment;

FIG. 4 illustrates a signal diagram of the multi-channel electric stimulator for a neural implant according to an exemplary embodiment; and

FIG. 5 is a flowchart sequentially illustrating the operation of the multi-channel electric stimulator for a neural implant according to an exemplary embodiment.

### DETAILED DESCRIPTION OF THE INVENTION

Reference will now be made in greater detail to an exemplary embodiment of the present invention, an example of which is illustrated in the accompanying drawings. Wherever possible, the same reference numerals will be used throughout the drawings and the description to refer to the same or like parts.

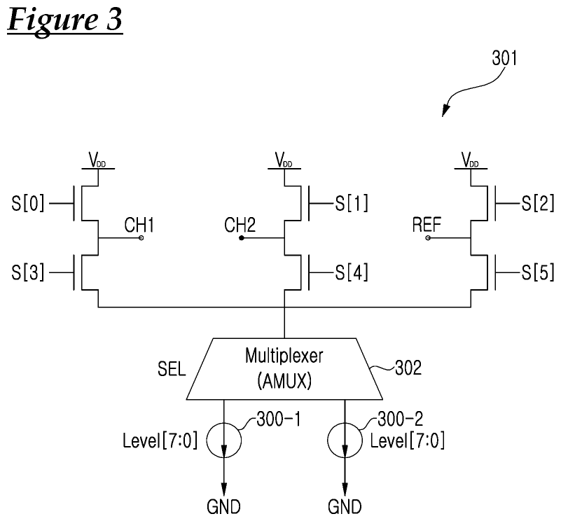

FIG. 3 is a circuit diagram illustrating a structure of a multi-channel electric stimulator for a neural implant according to an exemplary embodiment.

As illustrated in FIG. 3, the multi-channel electric stimulator according to an exemplary embodiment is a circuit realized on the basis of the concept of double buffering used in a digital circuit. The multi-channel electric stimulator has a structure provided with a double buffering digital-to-analog converter (DAC). Specifically, the multi-channel electric stimulator includes: a direct current (DC) generator 300-1, 300-2, and 302 for generating direct current according to nerve stimulation channels (or in a nerve stimulation channel-specific manner) using a double buffering DAC; a biphasic current pulse generator (BCG) 301 for generating a biphasic current pulse by switching the direct current; and a switching operation controller (not shown).

In addition, the DC generator 300-1, 300-2, and 302 according to an exemplary embodiment includes a multiplexer 302 to alternately and sequentially select DC-supplying operations according to the nerve stimulation channels. In addition, the DC generator 300-1, 300-2, and 302 includes a DAC circuit comprised of a plurality of DACs. Due to the DAC circuit comprised of the plurality of DACs, the DC generator 300-1, 300-2, and 302 has an operation of controlling a channel to stand by, with the electric current thereof modified into a DC level, when selected for a point in time, at which another pulse is generated, without being selected by the multiplexer 302, and supplying direct current at a point in time, at which a next stimulation pulse is generated.

The DC generator 300-1, 300-2, and 302 is configured to supply direct current in response to nerve stimulation in the sequence of stimulation by causing a nerve stimulation channel next in the sequence of stimulation among adjacent nerve stimulation channels to stand by, with the electric current of the next nerve stimulation channel being modified into a DC level, in a case of nerve stimulation using a nerve stimulation channel earlier in the sequence of stimulation among the adjacent nerve stimulation channels. Due to the DC generator 300-1, 300-2, and 302, DAC switching noise is not transferred to the circuit. The DC generator 300-1, 300-2, and 302 determines the magnitude of nerve stimulation current.

The BCG 301 generates biphasic current pulses by switching direct current according to the nerve stimulation channels. The BCG 301 includes DC switches to generate biphasic current pulses according to the nerve stimulation channels. In addition, the BCG generates biphasic current pulses by performing switching using the switches differently according to the nerve stimulation channels. Accordingly, the nerve is stimulated by the biphasic current pulses.

The controller (not shown) controls the switching operation of the BCG 301, in the sequence of stimulation, which is set according to the nerve stimulation channels.

In addition, the DC generator 300-1, 300-2, and 302 according to an exemplary embodiment illustrated in FIG. 3 is a circuit realized on the basis of the concept of double buffering. More specifically, the DC generator 300-1, 300-2, and 302 according to an exemplary embodiment includes a multiplexer to alternately and sequentially select DC-supplying operations in a set sequence of stimulation according to the nerve stimulation channels in a case of nerve stimulation according to the nerve stimulation channels. In addition, the DC generator 300-1, 300-2, and 302 includes DAC circuits 300-1 and 300-2, each of which is comprised of a plurality of DACs according to DC levels. In the DC generator 300-1, 300-2, and 302, each of the plurality of DACs of the DAC circuits 300-1 and 300-2 has an operation of standing by, with the electric current thereof being modified into a DC level, when selected for a point in time, at which another pulse is generated, without being selected by the multiplexer 302, and supplying the direct current at a point in time, at which a next stimulation pulse is generated, when selected.

In a case of nerve stimulation according to the nerve stimulation channels comprised of two channels, including a first channel that is first in the sequence of stimulation, and a second channel that is next in the sequence of stimulation, the multiplexer 302 connects the first channel to a DAC and supplies direct current to the first channel, with first priority according to the set sequence of stimulation. Here, when the multiplexer 302 is selected by another DAC, the second channel stands by, with the electric current thereof being previously modified into a DC level. Afterwards, the second channel is connected to the DAC, with second priority according to the set sequence of stimulation, and direct current is supplied to the second channel. In the same manner, when selected by the multiplexer 302, another DAC causes the first channel to stand by, with the electric current thereof being previously modified into a DC level. Accordingly, DC supplying operations are alternately and sequentially selected in the set sequence of stimulation, according to the nerve stimulation channels.

Each of the DAC circuits 300-1 and 300-2 includes a plurality of DACs according to DC levels. In addition, since each of the DAC circuits 300-1 and 300-2 is comprised of the plurality of DACs, when a channel is not selected by the multiplexer 302 and another pulse waveform is generated, the DAC circuits 300-1 and 300-2 cause the channel to stand by, with the electric current of the channel being modified into a level of direct current to be supplied at a point in time at which the channel will be selected. The point in time is a point in time at which another stimulation biphasic current pulse is generated according to an exemplary embodiment. Afterwards, when a next stimulation biphasic current pulse is generated, the DAC circuits 300-1 and 300-2 allow current to flow by determining the magnitude of current, due to the plurality of DACs. Consequently, DAC switching noise is not transferred to the circuit.

In addition, in the DAC according to an exemplary embodiment illustrated in FIG. 3, each of the plurality of DACs is provided as a double buffering DAC according to an exemplary embodiment.

Specifically, each of the plurality of DACs of the DAC circuits according to an exemplary embodiment includes a clock circuit (not shown) to generate a DC level change clock at a point in time, at which another pulse is generated, not selected by the multiplexer, and to generate a digital signal generation clock when selected. In addition, each of the DAC circuits according to an exemplary embodiment includes a digital terminal (not shown) that causes a channel to stand by, with the electric current of the channel being modified into a DC level, at the DC level change clock when selected by the multiplexer and converts direct current into an analog signal by determining the magnitude of the direct current and allowing the direct current to flow at the digital signal generation clock.

The clock circuit (not shown) generates a DC level change clock at a point in time, at which another stimulation biphasic current pulse is generated, without being selected by the multiplexer. In addition, fundamentally, the clock circuit (not shown) outputs a digital signal generation clock when selected by the multiplexer.

The digital terminal (not shown) synchronizes the direct current with the DC level change clock, so that the direct current stands by, with the direct current being modified to a DC level that is to be supplied when selected by the multiplexer. In addition, the digital terminal (not shown) synchronizes the direct current with the digital signal generation clock and causes the direct current to flow by determining the magnitude of the direct current. Consequently, the digital terminal (not shown) converts the direct current of the digital signal, generated as above, into an analog signal, in response to the switching of the BCG.

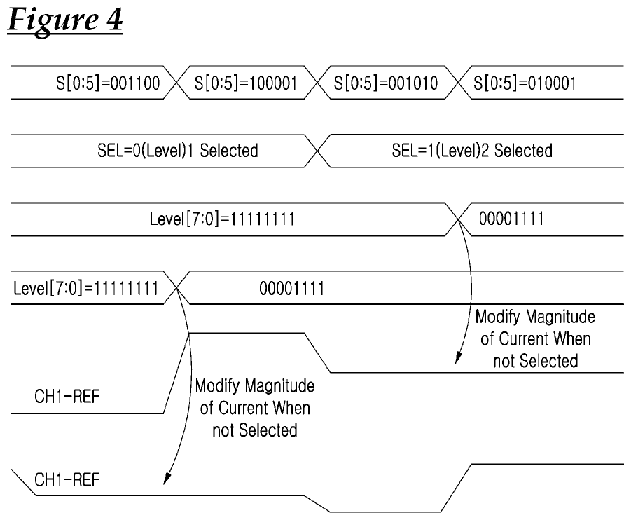

FIG. 4 illustrates a signal diagram of the multi-channel electric stimulator for a neural implant according to an exemplary embodiment.

As illustrated in FIG. 4, in the multi-channel electric stimulator for a neural implant according to an exemplary embodiment, a pulse waveform generated in a case in which none of two DACs connected to the multiplexer are selected is different from a pulse waveform generated in a last case before the case in which none of two DACs connected to the multiplexer are selected. In addition, when the different pulse waveform is generated, electric current is modified to a current level to flow at a selected point in time. Thus, DAC switching noise is not transferred to the circuit, since the magnitude of the electric current is modified before the standby operation, and the electric current is allowed to flow, with the magnitude thereof being determined, when a next stimulation pulse is generated. Here, for example, Level 1 means a current level in the last case before the case in which none of the DACs is selected in the first channel. In addition, in this case, the electric current is modified to a current level different from level 1, in response to channel switching, and the magnitude of the electric current is changed in the unselected case.

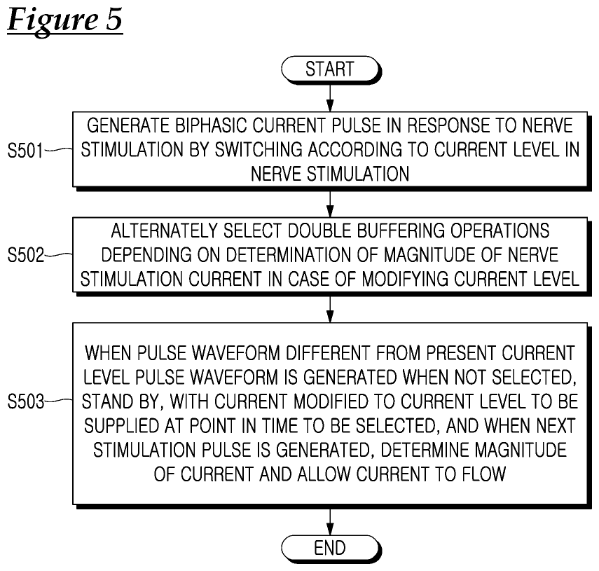

FIG. 5 is a flowchart sequentially illustrating the operation of the multi-channel electric stimulator for a neural implant according to an exemplary embodiment.

As illustrated in FIG. 5, in the electric stimulator according to an embodiment, the BCG differently switches a switching circuit corresponding to a nerve stimulation channel and a switching circuit corresponding to a nerve stimulation reference, depending on the intensity of nerve stimulation or the like, so that, when direct current is generated by the DAC, a biphasic current pulse induced by nerve stimulation is generated (S501).

In addition, in the case of nerve stimulation according to the nerve stimulation channels, the multiplexer alternately and sequentially selects DC supplying operations in the set sequence of stimulation according to the nerve stimulation channels (S502).

Then, when a channel is not selected by the multiplexer, the double-buffering two DAC circuits cause the channel to stand by, with the electric current of the channel being modified into a current level to be supplied at a point in time at which another stimulation biphasic current pulse is generated. In addition, when the channel is selected by the multiplexer, the DAC circuits allow direct current to flow by determining the magnitude of the direct current at a point in time at which a next stimulation biphasic current pulse is generated (S503). The above-described operations according an exemplary embodiment are performed whenever channels are changed. More particularly, the two DACs alternately repeats the operation of previously modifying the magnitude of electric current and the operation of supplying direct current by determining the magnitude of the D current in response to generation of a stimulation pulse. The operations performed as described above are based on the concept of double buffering according to an exemplary embodiment.

Accordingly, DAC switching noise is not transferred to the circuit.

In addition, a biphasic current pulse for nerve stimulation is generated by switching the above-described direct current, in response to the switching of the BCB.

Consequently, the nerve stimulated is, thereby replacing the function of a damaged nerve or restricting undesirable neural activity.

As set forth above, according to exemplary embodiments, in a case of nerve stimulation using a nerve stimulation channel earlier in the sequence of stimulation among adjacent nerve stimulation channels, a nerve stimulation channel next in the sequence of stimulation among the adjacent nerve stimulation channels stands by, with the electric current thereof being modified into a DC level, and direct current is supplied in response to nerve stimulation in the sequence of stimulation.

For example, when none of the two DACs connected to the multiplexer connected to the BCG is selected another pulse waveform is generated, electric current is modified to a current level to flow at a point in time of selection. Thus, the channel is caused to stand by, with the magnitude of electric current thereof being modified. When a next stimulation pulse is generated, the magnitude of electric current is determined and the electric current is allowed to flow, so that DAC switching noise is not transferred to the circuit. Accordingly, it is possible to reduce stimulation pulse intervals as small as possible in the case of continuous interleaved sampling (CIS) stimulation, thereby providing an electric stimulation circuit having a higher pulse rate. Thus, it is possible to reduce inter-channel intervals, thereby performing CIS stimulation using more channels in a predetermined time.

Although the exemplary embodiments of the present invention have been described for illustrative purposes, those skilled in the art will appreciate that various modifications, additions and substitutions are possible, without departing from the scope and spirit of the present invention as disclosed in the accompanying claims.

It is to be understood that the foregoing is a description of one or more preferred exemplary embodiments of the invention. The invention is not limited to the particular embodiment(s) disclosed herein, but rather is defined solely by the claims below. Furthermore, the statements contained in the foregoing description relate to particular embodiments and are not to be construed as limitations on the scope of the invention or on the definition of terms used in the claims, except where a term or phrase is expressly defined above. Various other embodiments and various changes and modifications to the disclosed embodiment(s) will become apparent to those skilled in the art. All such other embodiments, changes, and modifications are intended to come within the scope of the appended claims.

As used in this specification and claims, the terms “for example,” “e.g.,” “for instance,” “such as,” and “like,” and the verbs “comprising,” “having,” “including,” and their other verb forms, when used in conjunction with a listing of one or more components or other items, are each to be construed as open-ended, meaning that the listing is not to be considered as excluding other, additional components or items. Other terms are to be construed using their broadest reasonable meaning unless they are used in a context that requires a different interpretation.

What is claimed is:

1. A multi-channel electric stimulator for a neural implant, wherein the multi-channel electric stimulator switches direct current according to nerve stimulation channels of differently-set sequences of stimulation in a case of nerve stimulation, and comprising:
a direct current generator configured to supply direct current in response to nerve stimulation in a sequence of stimulation by controlling a nerve stimulation channel next in the sequence of stimulation among adjacent nerve stimulation channels to stand by, with an electric current of the next nerve stimulation channel being modified into a direct current level, when nerve stimulation is performed using a nerve stimulation channel earlier in the sequence of stimulation among the adjacent nerve stimulation channels;
a biphasic current pulse generator generating a biphasic current pulse by switching the nerve stimulation channel-specific direct current supplied thereto; and
a controller controlling a switching operation of the biphasic current pulse generator depending on a sequence of stimulation set according to the nerve stimulation channels.

2. The multi-channel electric stimulator according to claim 1, wherein the direct current generator comprises:
a multiplexer alternately and sequentially selecting direct current supplying operations in the sequence of stimulation according to the nerve stimulation channels in a case of nerve stimulation according to the nerve stimulation channels; and
a digital-to-analog converter circuit comprising a plurality of digital-to-analog converters according to direct current levels, wherein each of the plurality of digital-to-analog converter circuit stands by at a direct current level when selected for a point in time, at which another stimulation biphasic current pulse is generated, without being selected by the multiplexer, and supplies direct current at a point in time, at which a next stimulation biphasic current pulse is generated, when selected.

3. The multi-channel electric stimulator according to claim 2, wherein each of the plurality of digital-to-analog converters comprises a double buffering digital-to-analog converter comprising:
a clock circuit generating a direct current level change clock at a point in time, at which another stimulation biphasic current pulse is generated, without being selected by the multiplexer, and outputting a digital signal generation clock at a point in time, at which a next stimulation biphasic current pulse is generated, when selected by the multiplexer; and
a digital terminal synchronizing the direct current with the direct current level change clock so that the direct current to stand by, with the direct current being modified to a direct current level to be supplied when selected by the multiplexer, and synchronizing the direct current with the digital signal generation clock so that a magnitude of the direct current is determined and the direct current flows, thereby converting the direct current into an analog signal.
